(19) United States
(12) Patent Application Publication
(10) Pub. No.: US 2004/0014066 A1
(43) Pub. Date: Jan. 22, 2004

(54) Screening system

(76) Inventors: Hee-Jeon Hong, Norwich (GB); Mark Sebastian Boye Paget, Norwich (GB); Mark Jeremy Buttner, Norwich (GB)

Correspondence Address: Harness Dickey & Pierce, Suite 400, 7700 Bonhomme, St Louis, MO, 63105, US

(21) Appl. No.: 10/297,031

(22) PCT Filed: May 30, 2001
(86) PCT No.: PCT/GB01/02400

(30) Foreign Application Priority Data
Jun. 1, 2000 (GB) 0013384.3

(51) Int. Cl. C12Q 1/68; C12Q 1/18

(52) U.S. Cl. 435/6; 435/32

(57) ABSTRACT

The invention provides a method of detecting in a sample the activity of an antibiotic which affects cell integrity, which method comprises the steps of: a) providing a transformed microorganism which comprises a nucleic acid encoding a promoter operably linked to a heterologous reporter gene capable of causing a detectable signal; b) contacting the sample with the transformed microorganism; c) observing said microorganism for said detectable signal, wherein the promoter is regulated by a two-component signal transduction system, which signal components are: i) a receptor responsive to changes in the cell envelope or membrane of the microorganism, ii) a trans-acting factor which is activated in response to stimulation by the receptor, and which regulates the promoter. Preferred signal components are derived from CseB and CseC which regulate a sigE promoter. Also provided are corresponding microorganisms, systems, kits, and processes of use of these things.

### TECHNICAL FIELD

The present invention relates to methods and materials for screening for compounds which have potential as antibiotics. It further relates to methods for generating microorganisms having utility in screening, tools which can be generally used in such methods, the microorganisms themselves, and biosensing methods employing the microorganisms.

### BACKGROUND ART

There is an ongoing requirement for novel compounds that have antibiotic activity, for instance to counteract the problem of drug resistance. Methods for screening potential sources of antibiotic that have been used in the prior art include those which are based on particular ‘indicator’ or ‘reporter’ strains of bacteria.

For instance, the use of E. coli PG8 (lacking both chromosomal β-lactamase and the cell wall biosynthetic enzyme PBP 1B) as an indicator strain led to the discovery of the monocyclic β-lactams [the nocardicins (Aoki et al., 1976) and the monobactams (Imada et al., 1981)].

Another screening approach has exploited the fact that the β-lactamase of Bacillus licheniformis is induced by β-lactams; the β-lactamase produced is easily detected by a chromogenic reaction, and this reporter system led to the independent discovery of the monobactams (Sykes et al., 1981).

It can thus be seen that novel indicator systems would provide a contribution to the art.

### DISCLOSURE OF THE INVENTION

The present inventors have provided a novel system to provide a broad-range, generic screen for antibiotics, lytic enzymes and other compounds which target the cell envelope.

The system is based on the use of a promoter, linked to a suitable reporter gene, which promoter is regulated by a two-component signal transduction system. The two components are (i) a cell membrane located sensor, and (ii) a trans-acting factor which is activated in response to a stimulus to the sensor such that it induces transcription of the reporter gene linked to the promoter.

As a test system, the inventors have established that the promoter from the gene encoding σ^{E} (sigE) can be used as a generic detector of antibiotics or enzymes that interfere with the physical integrity of the cell envelope when tested in Streptomyces coelicolor A3(2).

It was already known that the gene encoding sigE was regulated at the level of transcription by a two-component signal transduction system (CseB and CseC) (Paget et al., 1999b). However, although the sigE transcription factor (σ^{E}) was believed to control certain cell wall-related functions in S. coelicolor A3(2) in response to changes in the cell envelope (Lonetto et al., 1994; Paget et al., 1999a; Paget et al., 1999b) the precise nature of the initiating signal was unknown. There was no suggestion in any of these publications that the system had utility as a screening agent.

Thus in a first aspect of the invention there is disclosed a method of detecting an activity of an antibiotic analyte in a sample comprising the steps of:

(a) providing a transformed microorganism which comprises a nucleic acid encoding a promoter which is regulated by a two-component signal transduction system operably linked to a heterologous reporter gene capable of causing a detectable signal,

(b) contacting the sample with the transformed microorganism,

(c) observing said bacterium for said detectable signal;

The term “antibiotic” is used broadly in this aspect to cover all antimicrobial compounds (natural, semi-synthetic or synthetic) which inhibit or kill (susceptible) microorganisms and to embrace compounds which interfere with the physical integrity of the cell envelope. Suitable antibiotics include but are not limited to penicillins (such as penicillin G, amoxycillin and ticarcillin), glycopeptides (such as teicoplanin, ristocetin and vancomycin) and peptides such as bacitracin. Glycopeptides and peptide antibiotics are preferred. As stated above, the method can also be used to detect activity of lytic enzymes. Where the term “antibiotic” is used hereinafter, the skilled person will appreciate that this applies mutatis mutandis to such enzymes.

The activity detected may be correlated with the presence or absence of an antibiotic, or putative antibiotic, in the sample in a qualitative manner. Alternatively it may be used to make a quantitative assessment.

As described above, the “two components” are (i) a sensor (e.g. receptor), which will be receptive to changes in the cell envelope or membrane of the microorganism (and will generally be membrane bound) and (ii) a trans-acting factor which is activated (e.g. phosphorylated) in response to stimulation of the sensor such that it activates the promoter and induces transcription of the reporter gene linked to the promoter. An example of such a system is shown in FIG. 1, which shows the sigE promoter, which is regulated by CseB and CseC.

The term “operably linked” refers to the linkage of a promoter to an RNA-encoding DNA sequence, and especially to the ability of the promoter to induce production of RNA transcripts corresponding to the DNA sequence when the promoter or regulatory sequence is recognized by a suitable polymerase. The term means that linked DNA sequences (e.g. promoter, reporter gene, terminator sequence) are operational or functional, i.e. work for their intended purposes.

By “observing” is meant ascertaining by any means (directly or indirectly) the presence or absence of the selected signal which is indicative of the binding event.

Thus the invention provides, inter alia, a method of detecting in a sample the activity of an antibiotic which affects cell integrity, which method comprises the steps of: (a) providing a transformed microorganism which comprises a nucleic acid encoding a promoter operably linked to a heterologous reporter gene capable of causing a detectable signal, (b) contacting the sample with the transformed microorganism, (c) observing said microorganism for said detectable signal, wherein the promoter is regulated by a two-component signal transduction system, which signal components are (i) a receptor responsive to changes in the cell envelope or membrane of the microorganism (ii) a trans-acting factor which is activated in response to stimulation by the receptor, and which regulates the promoter.

Some particular embodiments and aspects will now be discussed in more detail.

Choice of Sample

Samples may be selected from any suitable source. In particular, samples may be selected from culture supernatants and extracts from soil isolates, compounds produced by chemical synthesis including combinatorial chemistry; and compounds produced by combinatorial biosynthesis.

Also provided is a process of producing an isolated antibiotic which affects cell integrity, which method comprises the steps of: (a) performing a method as described above such as to identify the activity of the antibiotic in a sample, (b) isolating the antibiotic from the sample.

Choice of Promoter

In one embodiment, the promoter is the sigE promoter or an active variant thereof. The nucleotide sequence of the sigE operon is published (Paget et al., 1999b).

‘Variants’ in this context will have promoter activity, by which is meant the ability to bind an RNA polymerase (and other factors that initiate or modulate transcription under the e.g. sigE promoter) whereby an RNA transcript is produced from the reporter gene under the appropriate conditions i.e. activation of the component signal transduction pathway.

Variants may be modified from an ‘authentic’ native sequence e.g. by introducing changes into the full-length or part-length sequence, for example substitutions, insertions, and/or deletions. This may be achieved by any appropriate technique, including restriction of the sequence with an endonuclease followed by the insertion of a selected base sequence (using linkers if required) and ligation. Also possible is PCR-mediated mutagenesis using mutant primers.

It may, for instance, be preferable to add in or remove restriction sites in order to facilitate further cloning. Modified sequences according to the present invention may have a sequence at least 70% identical to the sequence of the full or part-length inducible promoter or operon protein as appropriate. Typically there is 80% or more, 90% or more 95% or more or 98% or more identity between the modified sequence and the authentic sequence. There may be up to five, for example up to ten or up to twenty or more nucleotide deletions, insertions and/or substitutions made to the full-length or part length sequence provided functionality is not totally lost.

Similarity or identity may be as defined and determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using FASTA and FASTP (see Pearson & Lipman, 1988. Methods in Enzymology 183: 63-98). Parameters are preferably set, using the default matrix, as follows:

Gapopen (penalty for the first residue in a gap): −16 for DNA

Gapext (penalty for additional residues in a gap): −4 for DNA

KTUP word length: 6 for DNA.

Alternatively, homology in this context can be judged by probing under appropriate stringency conditions. One common formula for calculating the stringency conditions required to achieve hybridization between nucleic acid molecules of a specified sequence homology is (Sambrook et al., 1989):

Tₘ=81.5° C.+16.6 Log[Na+]+0.41 (% G+C)−0.63 (% formamide) 600/#bp in duplex.

As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

A system may be based on two-component signal transduction systems other than the sigE operon. For instance another system that has been proposed to sense and respond to physical changes in the cell envelope is the VanS/VanR two-component system which activates transcription from the vanH promoter (vanHp) in Enterococcus faecium. This system is described by Ulijasz et al. (1996). However there is no suggestion therein of its utility in the methods of the present invention. Another system is the the KdpD/KdpE sensor kinase/response regulator pair of E. coli which responds to decreases in extracellular K⁺ concentration as well as increases in medium osmolarity. This system is described by Walderhaug et al (1992) and Sugiura et al (1994).

Use of the promoters discussed above, particularly the sigE promoter or an active variant thereof, in the methods or systems described herein, forms one aspect of the present invention.

Choice of Reporter Gene

Generally the observed signal arises in consequence to an increased expression of a reporter protein from the reporter gene.

In the examples herein, the reporter protein provides resistance against a particular antibiotic (or otherwise lethal concentration of antibiotic). The detection in this case is the viability and/or increased rate of growth of the host microorganism in the presence of the antibiotic. This can be visualised e.g. directly, as a bacterial lawn on a nutrient plate. An example is the neo gene which confers resistance to both neomycin and kanamycin.

Alternative reporter genes may be used for increased ease of scoring and/or sensitivity. Most preferably the activity of the signal protein, or the protein itself, can be estimated photometrically (especially by fluorimetry or luminometry). This may be directly e.g. using instance green (and red) fluorescent protein, insect luciferase, and photobacterial luciferase. Alternatively it may be indirect e.g. whereby the signal gene causes a change which is detected by a colour indicator e.g. a pH change. In particular, the lux genes of Vibrio harveyi have been used successfully in Streptomyces.

Other suitable signal proteins (which have a readily detectable activity) are known in the art e.g. β-galactosidase, which can generate a coloured substrate. The signal may utilise co-factors.

Methods for introducing signal genes into appropriate hosts are described in further detail below.

Integration of Reporter Gene

In one embodiment, the reporter gene may be introduced into the host such that it is operably linked to an appropriate existing inducible promoter. Typically this will be achieved by initiating targeted integration using aspects of the sequence forming part of the promoter region or operon. Direct integration of a signal gene system such as luciferase (e.g. luxAB operon) into an environmentally responsive regulon in the host microorganism can be achieved by transposition or by illegitimate or legitimate recombination between a genetic construct introduced into the cell and the target operon or gene cluster located on either the chromosome or an episomal element.

In such cases, although the promoter is native to the cell, the reporter gene is “heterologous” to the promoter, by which is meant that the gene in question has been introduced into the cell using genetic engineering, i.e. by human intervention. Generally the heterologous gene will be non-naturally occurring in cells of that type.

Nucleic Acid Constructs

Preferably the nucleic acid encoding the promoter operably linked to the heterologous reporter gene capable of causing a detectable signal is in the form of an extrachromosomal vector.

“Vector”, unless further specified, is defined to include, inter alia, any plasmid DNA, lysogenic phage DNA and/or transposon DNA, in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication).

Strain derivatives encoding different gene dosage levels of the promoter/signal gene can be created by integration of the construct into the chromosome (low copy number/low sensitivity) or by use of medium or high copy number plasmids (medium or high sensitivity).

One example herein is based on the neo reporter plasmid, pIJ486, which was published in 1986 (Ward et al., 1986) and has been used widely in the Streptomyces community to identify promoter-containing fragments and to quantify the strength of promoters in vivo. It has also been used to identify compounds which induce given promoters (e.g. Salah-Bey et al., 1995; Murakami et al., 1989). This has been used to generate the sigEp-neo fusion plasmid (pIJ6880) as described hereinafter.

Generally speaking, those skilled in the art are well able to construct vectors and design protocols for recombinant gene expression in common bacterial hosts. Suitable vectors can be chosen or constructed, containing appropriate regulatory sequences, including promoter sequences, terminator fragments, polyadenylation sequences, enhancer sequences and other sequences as appropriate. For further details see, for example, Molecular Cloning: a Laboratory Manual: 2nd edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

Vectors for use in the invention will typically include: (a) a promoter which is regulated by a two-component signal transduction system operably linked to, (b) a heterologous reporter gene capable of causing a detectable signal, wherein the “two components” are (i) a sensor, which will be receptive to changes in the cell envelope or membrane of the microorganism and (ii) a trans-acting factor which is activated in response to stimulation of the sensor such that it activates the promoter.

In one assay described hereinafter, the sigE promoter is on a plasmid replicon with a copy number of 100-200 per chromosome. However the gene (cseB) encoding the activator of the promoter is present in single copy in the chromosome. In order to improve sensitivity it may be desired to increase the copy number of all or some of the genes encoding the two component system, which will generally be part of the promoter's native operon. For instance, in the assay employed in the examples below, additional copies of cseb may enhance the sensitivity of the assay. Similarly, changing the copy number of the sensor kinase gene (cseC) or of cseA (which is believed to function as a negative regulator of the system) might have this effect.

One way of increasing copy number would be for the operon genes(optionally modified) to be included in a vector, optionally operably linked to a strong promoter or even the inducible promoter. The vector may or may not be the same vector as that carrying the inducible promoter and reporter gene.

Vectors as described above form one aspect of the present invention, as do methods of using them to produce a transformed host cell. The vector may remain discrete in the host. Alternatively it may integrate into the genome of the host.

Choice of Host Strain

The assay may use any suitable species of bacteria. Preferably the assay uses a strain of Streptomyces e.g. M600, which is a plasmid-free derivative of wild-type S. coelicolor A3(2). The sigEp-neo fusion plasmid (pIJ6880) may then be introduced into the strain in question in order to perform the invention.

However it may be preferred to use other Streptomyces species that may have greater sensitivity to cell wall-specific antibiotics. For instance, specialised S. coelicolor host mutants may be employed that are defective in the cell wall and therefore hypersensitive to cell wall-specific antibiotics.

In addition, it may be preferred to use strains in which enzymes which may otherwise degrade antibiotics (thereby reducing the sensitivity of the assay) have been inactivated.

For example, it is known that S. coelicolor produces one or more β-lactamases (enzymes that inactivate β-lactams), and construction of a specialised host in which the β-lactamase structural genes have been inactivated would increase sensitivity of the bioassay in detecting β-lactams. The availability of the S. coelicolor genome sequence (at the ‘sanger.ac.uk/Projects/S_{—}coelicolor’ website) means that the identification of host genes for disruption is straightforward; for example, there are putative β-lactamase structural genes on cosmids H63 and J11. Similarly, there is a putative vancomycin resistance locus on cosmid 66T3, and disruption of this locus might increase sensitivity of the bioassay in detecting vancomycin-related glycopeptides.

It may also be preferred to use genera of bacteria in which the promoter employed in the invention is non-native. This may permit different spectra of inducing compounds to be revealed. In such cases it may be required to express other heterologous genes in the bacteria in order to ensure the functionality of the two-component assay system. Generally the other genes may include transport and binding proteins, as well as the sensor and trans-acting factor(s).

As described above, the operon could be present on the same vector as that carrying the promoter and reporter gene, if preferred.

Examples of suitable bacteria may include, for example, the mycobacteria which are closely related to the streptomycetes, and include important human pathogens such as Mycobacterium tuberculosis and Mycobacterium leprae. The four-gene sigE operon (in single copy) and the sigEp-neo fusion (in multicopy) could be expressed in Mycobacterium smegmatis to permit screening therein.

Preferably, however, the host microorganism is the same species as that which provided the source of the inducible promoter, and will therefore naturally express the other components of the system required to give screening function.

The host cells described above, which may include vectors of the present invention, or heterologous reporter genes, form a further aspect of the present invention. An example transformed microorganism for use in a method described above would be one which is transformed with a vector comprising (a) a promoter operably linked to (b) a heterologous reporter gene capable of causing a detectable signal, wherein the promoter is regulated by a two-component signal transduction system, which signal components are (i) a receptor responsive to changes in the cell envelope or membrane of the microorganism (ii) a trans-acting factor which is activated in response to stimulation by the receptor, and which regulates the promoter.

Systems

In a further aspect of the present invention there is provided a system for detecting an activity of an antibiotic in a sample comprising:

(a) a transformed microorganism as described above,

(b) means for detecting the signal produced from the reporter gene.

For example, where the signal is bacterial luciferase, this may be detected extracellularly using a photomultiplier or photodiode or any other photosensitive device.

Where the reporter gene encodes an antibiotic resistance gene (e.g. the neo gene in the Examples below) this could be used in parallel processing system in which assays for multiple potential inducers are assessed using multiple nutrient plates. Alternatively, using an automated 96-well microtitre dish assay, kanamycin-resistant growth may be assayed automatically by optical density.

Naturally the methods and systems of the invention described above could be used as a primary screen, with further screens (e.g. based on antibiosis of target organisms, which may be different species to the screening microorganism) being employed to further exclude compounds not having the desired activity.

Kits

Also embraced within the scope of the present invention are kits for performing the various aspects of the invention. For instance a kit suitable for use in the first aspect may comprise a preparation of the microorganism, plus further means for carrying out the contact or observation steps e.g. buffers, co-factors (e.g. luciferin for addition to luciferase).

The invention will now be further described with reference to the following non-limiting Figures and Examples. Other embodiments falling within the scope of the present invention will occur to those skilled in the art in the light of these.

### FIGURES AND TABLES

FIG. 1 shows a model for regulation of the sigE promoter in response to signals from the cell envelope.

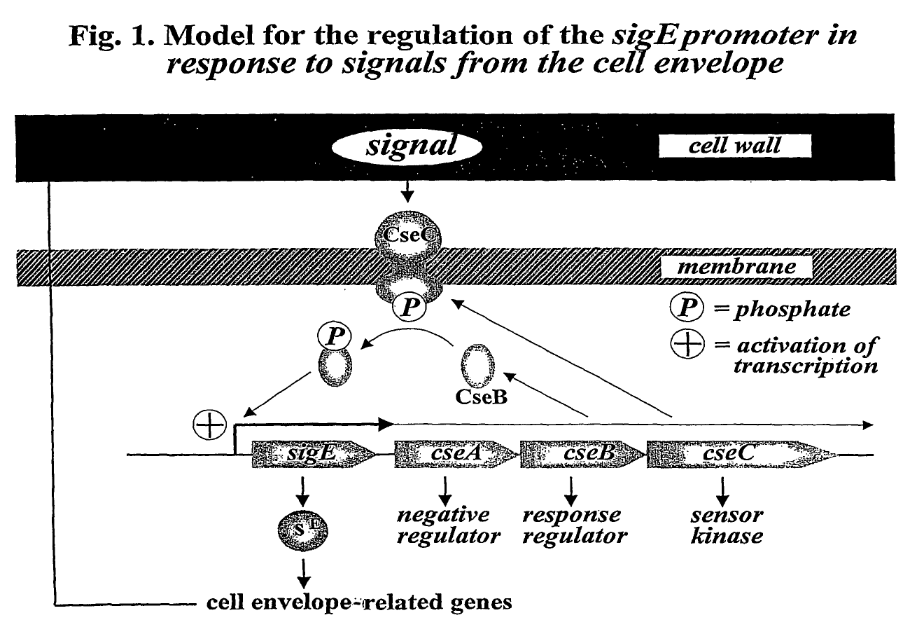

FIG. 2 shows induction of the sigE promoter by cell wall-specific antibodies.

Table 1 summarises the results of the experiment described in Example 2, in which 20 antibiotics were tested for ability to induce the sigE promoter.

### EXAMPLES

### Example 1

Construction of Reporter Construct

A ˜750 bp PvuII-SmaI fragment carrying the sigE promoter (sigEp), was blunt-end cloned into pIJ2925 (Janssen and Bibb, 1993) cut with SmaI and EcoRI to create pIJ5953.

The sigEp fragment was re-isolated from pIJ5953 as a ˜750 bp BglII fragment and cloned into the multicopy promoter probe plasmid pIJ486 (Ward et al., 1986) cut with BamHI, such that expression of the vector aminoglycoside phosphotransferase gene (neo), which confers resistance to both neomycin and kanamycin, depends on sigEp. The orientation of the insert was determined by digestion with SphI. The resulting plasmid was designated pIJ6880.

### Example 2

Construction and Use Testing of Reporter Strain

Plasmid pIJ6880 was introduced by protoplast transformation (Hopwood et al., 1985) into M600 (Chakraburtty and Bibb, 1997), a plasmid-free derivative of wild-type S. coelicolor A3(2), and was found to confer resistance to approximately 80 μg ml⁻¹ kanamycin (on MMT medium; Katz et al., 1983).

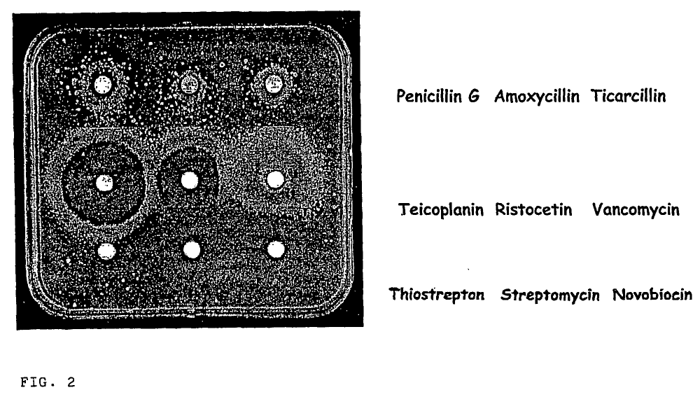

To see if the sigE promoter could be induced by control-antibiotics known to target the cell envelope, spores of M600 carrying pIJ6880 were spread on MMT medium carrying a lethal concentration of kanamycin (100 μg ml⁻¹) and potential inducers were applied on paper discs to the freshly spread plates. The results are shown for nine antibiotics in FIG. 2. Inducers of the sigE promoter (penicillin G, amoxycillin, ticarcillin, teicoplanin, ristocetin and vancomycin) raised the level of expression of the neo gene and hence induced a halo of kanamycin-resistant growth around the disc. The glycopeptides teicoplanin, ristocetin and vancomycin (and the peptide bacitracin; data not shown) were particularly potent inducers. In contrast, ‘negative control’ antibiotics that target the ribosome (e.g. thiostrepton, streptomycin) or DNA gyrase (novobiocin) do not induce a halo.

Table 1 summarises the results of the assay for the antibiotics used in FIG. 2 and for a further 11 antibiotics tested (✓=induced a halo of kanamycin resistant growth, indicating ability to induce the sigEp-neo fusion; X=did not induce a halo of kanamycin resistant growth). A wide range of penicillins (e.g. penicillin G, oxacillin, ampicillin, ticarcillin), cephalosporins (e.g. cefaclor, cefadroxil, cephradine, cephalexin), glycopeptides (e.g. teicoplanin, ristocetin, vancomycin) and peptides (bacitracin) were found to induce the sigE promoter (Table 1). Thus it is clear that this sigE promoter bioassay detects structurally unrelated antibiotics with varied targets in the cell envelope, allowing the system to provide a broad-range, generic screen for cell envelope-specific antibiotics.

Although it is still unknown precisely which physical characteristic of the cell envelope the CseB/CseC signal transduction system responds too, these results clearly show the utility of the system as a screen. For instance, an initial assessment of sensitivity demonstrated that 75 ng of ristocetin gives a positive reaction in the bioassay.

It should be noted that many of the antibiotics acted as inducers without showing any signs of toxicity (e.g. vancomycin, FIG. 2). With such antibiotics, the induced kanamycin-resistant halo grows to the edge of the antibiotic disc. The cell wall degradative enzyme lysozyme also acted as an inducer (data not shown), demonstrating that the sigE promoter bioassay can also be used to detect lytic enzymes that target the cell envelope.

TABLE 1

| | | Cephalosporins and | | | | | | ‘Negative Control’ antibiotics that | |
|---|---|---|---|---|---|---|---|---|---|
| Penicillins | | other β-lactams | | Glycoproteins | | Peptides | | do not target the cell envelope | |
| Penicillin G | ✓ | Cefaclor | ✓ | Teicoplanin | ✓ | Bacitracin | ✓ | Novobiocin (target-DNA gyrase) | X |
| Penicillin V | ✓ | Cefadroxil | ✓ | Ristocetin | ✓ | | | Thiostrepton (target-the ribosome) | X |
| Amoxycillin | ✓ | Cephapirin | ✓ | Vancomycin | ✓ | | | Streptomycin (target-the ribosome) | X |
| Ampicillin | ✓ | Cepharadine | ✓ | | | | | | |
| Mezlocillin | ✓ | Cefatrizine | ✓ | | | | | | |
| | | propylene glycol | | | | | | | |
| Piperacillin | ✓ | Cephazolin | ✓ | | | | | | |
| Ticarcillin | ✓ | Cefepime | ✓ | | | | | | |
| | | Cephalosporin | ✓ | | | | | | |
| | | Cephalexin | ✓ | | | | | | |

20 antiobiotics known to target the cell envelope and three ‘negative control’ antibiotics that do not target the cell envelope were tested for their ability to induce the sigEpo-neo fusion in the bioassay (✓ = induced a halo of kanamycin-resistant growth; X = did not induce a halo of kanamycin-resistant growth).
Nine examples of the primary data are shown in FIG. 2.
The glycopeptides, teicoplanin, ristocetin and vancomycin, and the peptide bacitracin were particularly potent inducers.

### Example 3

Use of Reporter Strain in Bioassay

In order to perform the assay of the invention,spores of the host microorganism, for example, M600 carrying pIJ6880, are spread on MMT medium carrying a lethal concentration of kanamycin (100 μg ml⁻¹)at concentration of approximately 5×10⁶/12 cm² plate. Test compound is applied on paper discs to a number of freshly spread plates in parallel using a different concentration of the test compound in each plate. A halo of kanamycin-resistant growth indicates that the test compound represents a cell envelope-specific antibiotic.

### References

Aoki et al. (1976) Nocardicin A, a new monocyclic β-lactam antibiotic. I. Discovery, isolation and characterisation. J. Antibiotics. 29: 492-500.

Chakraburtty, R. and Bibb, M. J. (1997) The ppGpp synthetase gene (relA) of Streptomyces coelicolor A3(2) plays a conditional role in antibiotic production and morphological differentiation. J Bacteriol 179: 5854-5861.

Hopwood, D. A., Bibb, M. J., Chater, K. F., Kieser, T., Bruton, C. J., Kieser, H. M., Lydiate, D. J., Smith, C. P., Ward, J. M., and Schrempf, H. (1985) Genetic Manipulation of Streptomyces: A Laboratory Manual. Norwich: The John Innes Foundation.

Imada, A., Kitano, K., Kintana, K., Muroi, M., and Asai, M. (1981) Sulfazecin and isosulfazecin, novel β-lactam antibiotics of bacterial origin. Nature 289: 590-591.

Janssen, G. R., and Bibb, M. J. (1993) Derivatives of pUC18 that have BglII sites flanking a multiple cloning site and that retain ability to identify recombinant clones by visual screening of Escherichia coli colonies. Gene 124: 133-134.

Katz, E., Thompson, C. J., and Hopwood, D. A. (1983) Cloning and expression of the tyrosinase gene from Streptomyces antibioticus in Streptomyces lividnas. J Gen Microbiol 129: 2703-2714.

Lonetto, M. A., Brown, K. L., Rudd, K. E., and Buttner, M. J. (1994) Analysis of the Streptomyces coelicolor sigE gene reveals the existence of a subfamily of eubacterial RNA polymerase a factors involved in the regulation of extracytoplasmic functions. Proc Natl Acad Sci USA 91: 7573-7577.

Murakami, T., Holt, T. G., and Thompson, C. J. (1989) Thiostrepton-induced gene expression in Streptomyces lividans. J Bacteriol 171: 1459-1466.

Paget, M. S. B., Chamberlin, L. Atrih, A., Foster, S. J. and Buttner, M. J. (1999a) Evidence that the ECF sigma factor, σ^{E}, is required for normal cell wall structure in Streptomyces coelicolor A3(2). J Bacteriol 181: 204-211.

Paget, M. S. B., Leibovitz, E., and Buttner, M. J. (1999b) A putative two-component signal transduction system regulates σ^{E}, a sigma factor required for normal cell wall integrity in Streptomyces coelicolor A3(2). Mol. Microbiol. 33: 97-107.

Salah-Bey, K., Blanc, V., and Thompson, C. J. (1995) Stress-activated expression of a Streptomyces pristinaespiralis multidrug resistance gene (ptr) in various Streptomyces spp. and Escherichia coli. Mol. Microbiol. 17: 1001-1012.

Sykes, R. B et al. (1981) Monocyclic β-lactam antibiotics produced by bacteria. Nature 291: 489-491.

Sugiura, A., Hirokawa, K., Nakashima, K., and Mizuno, T. (1994) Signal-sensing mechanisms of the putative osmosensor KdpD in Escherichia coli. Mol Microbiol 14: 929-938.

Ulijasz, A. T., Grenader, A., and Weisblum, B. (1996) A vancomycin-inducible lacz reporter system in Bacillus subtilis: induction by antibiotics that inhibit cell wall synthesis and by lysozyme. J Bacteriol 178: 6305-6309.

Walderhaug, M. O., Polarek, J. W., Voelkner, P., Daniel, J. M., Hesse, J. E., Altendorf, K., and Epstein, W. (1992) KdpD and KdpE, proteins that control expression of the kdpABC operon, are members of the two-component sensor-effector class of regulators. J Bacteriol 174: 2152-2159.

Ward, J. M., Janssen, G. R., Kieser, T., Bibb, M. J., Buttner, M. J. and Bibb, M. J. (1986) Construction and characterisation of a series of multi-copy promoter-probe plasmid vectors for Streptomyces using the aminoglycoside phosphotransferase gene from Tn5 as indicator. Mol. Gen. Genet. 203: 468-478.

1. A method of detecting in a sample the activity of an antibiotic which affects cell integrity, which method comprises the steps of:
(a) providing a transformed microorganism which comprises a nucleic acid encoding a promoter operably linked to a heterologous reporter gene capable of causing a detectable signal,
(b) contacting the sample with the transformed microorganism,
(c) observing said microorganism for said detectable signal, wherein the promoter is regulated by a two-component signal transduction system, which signal components are (i) a receptor responsive to changes in the cell envelope or membrane of the microorganism (ii) a trans-acting factor which is activated in response to stimulation by the receptor, and which regulates the promoter.

2. A method as claimed in claim 1 wherein the signal components are derived from CseB and CseC which regulate a sigE promoter.

3. A method as claimed in claim 1 wherein the signal components are derived from VanS and VanR which regulate the vanH promoter.

4. A method as claimed in claim 1 wherein the signal components are derived from KdpD and KdpE

5. A method as claimed in any one of the preceding claims wherein the reporter gene encodes a reporter protein which provides the microorganism with resistance to an antibiotic.

6. A method as claimed in any one of claims 1 to 4 wherein the reporter gene encodes a reporter protein which can be detected photometrically.

7. A method as claimed in any one of the preceding claims wherein the promoter encoded by the nucleic acid is native to the microorganism or the species of the microorganism.

8. A method as claimed in any one of the preceding claims wherein the nucleic acid is in the form of an extrachromosomal vector.

9. A method as claimed in any one of the preceding claims wherein the some or all of the genes encoding the two component system are present at enhanced copy number in the transformed microorganism relative to the corresponding untransformed microorganism.

10. A method as claimed in any one of the preceding claims wherein some or all of the genes encoding the two component system are operably linked to a strong promoter such as to enhance their concentration in the microorganism.

11. A method as claimed in any one of the preceding claims wherein the microorganism is selected such as to have an enhanced sensitivity to cell wall-specific antibiotics.

12. A method as claimed in any one of the preceding claims wherein the microorganism is a bacterium.

13. A method as claimed in claim 12 wherein the bacterium is selected from the following species: Streptomyces; Mycobacteria.

14. A method as claimed in any one of the preceding claims wherein the sample is selected from: a culture supernatant; a soil isolate; the product of combinatorial chemical synthesis; the product of combinatorial biosynthesis.

15. A method as claimed in any one of the preceding claims wherein the activity is qualitatively correlated with the presence or absence of an antibiotic.

16. A method as claimed in any one of the preceding claims wherein the activity of the sample is further screened for antibiosis of a target organism.

17. A process of producing a transformed microorganism for use in any one of the preceding claims, which process comprises transforming a microorganism with a vector comprising (a) a promoter operably linked to (b) a heterologous reporter gene capable of causing a detectable signal, wherein the promoter is regulated by a two-component signal transduction system, which signal components are (i) a receptor responsive to changes in the cell envelope or membrane of the microorganism (ii) a trans-acting factor which is activated in response to stimulation by the receptor, and which regulates the promoter.

18. A process as claimed in claim 17 wherein the copy number of the genes encoding the two component system is enhanced in the transformed microorganism relative to the corresponding untransformed microorganism.

19. A process of producing an isolated antibiotic which affects cell integrity, which method comprises the steps of:
(a) performing a method according to any one of claims 1 to 16 such as to identify the activity of the antibiotic in a sample,
(b) isolating the antibiotic from the sample.

20. A process as claimed in claim 19 which is preceded by the step of providing a transformed microorganism according to claim 17 or claim 18.

21. A transformed microorganism for use in a method of any one of claims 1 to 16, which microorganism is transformed with a vector comprising (a) a promoter operably linked to (b) a heterologous reporter gene capable of causing a detectable signal, wherein the promoter is regulated by a two-component signal transduction system, which signal components are (i) a receptor responsive to changes in the cell envelope or membrane of the microorganism (ii) a trans-acting factor which is activated in response to stimulation by the receptor, and which regulates the promoter.

22. A transformed microorganism as claimed in claim 21 wherein the copy number of the genes encoding the two component system is enhanced in the transformed microorganism relative to the corresponding untransformed microorganism.

23. A system for detecting an activity of an antibiotic in a sample comprising:
(a) the transformed microorganism of claim 21 or claim 22,
(b) means for detecting the signal produced from the reporter gene.

24. A system as claimed in claim 23 wherein the means are a photosensitive device.

25. A system as claimed in claim 23 or claim 24 which is a parallel processing system in which detection of multiple activities is assessed using multiple cultures of transformed microorganisms.

26. A kit for performing a method according to any one of claims 1 to 16, which kit comprises a preparation of the microorganism of claim 21 or claim 22, plus further means for carrying out the contact or observation steps.
